(19) United States
(12) Patent Application Publication
(10) Pub. No.: US 2023/0402187 A1
(43) Pub. Date: Dec. 14, 2023

(54) METHOD AND SYSTEM FOR MITIGATING PHYSICAL RISKS IN AN IOT ENVIRONMENT

(71) Applicant: SAMSUNG ELECTRONICS CO., LTD., Suwon-si (KR)

(72) Inventors: Niraj KUMAR, Dist-Giridih (IN); Bhiman Kumar BAGHEL, Korba (IN)

(73) Assignee: SAMSUNG ELECTRONICS CO., LTD., Suwon-si (KR)

(21) Appl. No.: 18/202,687

(22) Filed: May 26, 2023

Related U.S. Application Data
(63) Continuation of application No. PCT/KR23/06091, filed on May 4, 2023.

(30) Foreign Application Priority Data
Jun. 13, 2022 (IN) 202241033777

(51) Int. Cl. G16H 50/30; G16H 40/60

(52) U.S. Cl. CPC G16H 50/30; G16H 40/60

(57) ABSTRACT

A method for mitigating physical risks associated with a user in an Internet of Things (IoT) environment includes monitoring multi-modal input data associated with at least one multi-modal interaction of the user with at least one IoT device, identifying a change in at least one cognitive ability of the user, estimating a cognitive ability index of the user, predicting at least one physical risk associated with a current user activity, determining at least one corrective action to avoid the at least one physical risk, and controlling at least one IoT device to notify or perform the at least one corrective action such that the at least one physical risk is mitigated.

### CROSS-REFERENCE TO RELATED APPLICATION(S)

This application is a bypass continuation of PCT International Application No. PCT/KR2023/006091, filed on May 4, 2023, which claims priority to Indian Patent Application No. 202241033777, filed on Jun. 13, 2022, in the Indian Patent Office, the disclosures of which are incorporated herein by reference in their entireties.

### BACKGROUND

### 1. Field

The disclosure relates to the field of artificial intelligence, and more specifically, to a method and system for mitigating physical risks associated with a user in an Internet of Things (IoT) environment.

### 2. Description of Related Art

In recent years monitoring systems with new technologies are becoming of great concern to countries all over the world. IoT is an emerging technology that consists of various sensors and monitoring devices which are necessary tools for IoT-based smart environments. Among the various applications that the IoT facilitated to the world, one such important application is continuous monitoring of an individual's cognitive and physical health.

The monitoring devices monitor the individual's cognitive and physical health and understand the everyday activities of the individuals to gain insights that affect the quality of life. Everyday activities encompass a range of daily functional abilities that the individuals must complete to live competently and independently such as, but not limited to, cooking, bathing, reading, driving, and indoor activities. However, the monitoring devices of the related art only monitor the external appearances of the face and body of the individuals and do not provide the correct indication about the risk of accidents in the Smart home environment. For example: in medical science “Smiling Depression” is a term for someone living with depression on the inside while appearing perfectly happy or content on the outside. Their public life is usually one that's “put together,” maybe even what some would call normal or perfect. Similarly, all other information collected by using wearable or non-wearable devices can misguide us.

In particular, the monitoring devices of the related art are unable to predict an accidental risk due to cognitive decline in the ability of the user to perform the aforementioned activities. For example, when a user performs basic food cooking activities in a kitchen area, the user may meet with burning accidents due to a lack of attention, a compromised hand grip, other cognitive impairments, or lack of decision making. Similarly, when the user uses electronic appliances for cooking, the user may meet with an accident due to a lack of attention, loss of ability to organize tasks, emotional imbalance, or due to some other physical and mental conditions caused by cognitive decline. In another example, when the user working in the kitchen has some food or cooking item in hand, the user may fall down in the kitchen while working with kitchen equipment due to physical and mental conditions caused by the cognitive decline. Further, the user activities in the kitchen like chopping of vegetables, fruits, or salads require a high level of alertness, and a decline in physical, mental, and similar status (caused by cognitive decline), may result in a saviour accident.

In another example scenario with respect to bathroom activities, the user may lose control in the washroom or toilet and accidents like falling on the floor may happen due to some cognitive decline issues. Even the user may fall down in a leaving area or other areas due to imbalance or loss of control caused by the cognitive decline.

In yet another example, in a case where the user's health condition is declining the user may not make a call or inform others for help due to a decline in the cognitive health. The user may even face worsening of the health situation due to a combination of one or more health issues and may not be able to call anyone for help or even may not be able to do the self-help. Also, for example, when the user suffers from a rapid depression-related condition, then an increase in the depression level of the user may make him unable to call for help.

Several times, old age people, sick people, or people having some mild or initial cognitive impairments are not able to analyze their actual physical and mental capabilities to perform the aforementioned user activities that require some level of fitness. Due to the cognitive impairments, the user may meet with an accident at these places, and even several times the user may find himself or herself unable to self-help or perform a call to inform someone for help. Further, during monitoring of the cognitive health or physical health of the user, an outlook of any person does not necessarily provide sufficient information about the user's health status.

It is evident from the above-discussed examples that the type of accidents may differ and even the severity of the accidents may also differ in accordance with the types of user activities. IoT-based monitoring devices of the related art are unable to predict the severity and the type of accident that may happen in real-time while performing the user activities within an indoor environment (e.g., smart home environment).

Therefore, there lies a need for a method and system that can automatically reduce a risk of an accident and may control the damage by intelligently helping the user in case of accidents. Accordingly, the present disclosure provides a method and system for mitigating physical risks associated with a user while performing an activity or task in the IoT environment.

### SUMMARY

According to an aspect of the disclosure, a method for mitigating physical risks associated with a user in an Internet of Things (IoT) environment, includes: monitoring multi-modal input data over a first time period, the multi-modal input data being associated with at least one multi-modal interaction of the user with at least one IoT device in the IoT environment; identifying a change in at least one cognitive ability of the user, based on the monitored multi-modal input data; estimating a cognitive ability index of the user, based on the identified change in the at least one cognitive ability of the user; predicting at least one possible physical risk associated with a current user activity in the IoT environment based on a correlation of the estimated cognitive ability index with at least one cognitive health index from a plurality of cognitive health indexes; determining at least one corrective action to avoid the predicted at least one possible physical risk associated with the current user activity; and controlling at least one IoT device in the environment to notify or perform the determined at least one corrective action such that the predicted at least one possible physical risk is mitigated.

The method may include correlating the estimated cognitive ability index with the at least one cognitive health index; and predicting the at least one possible physical risk associated with the current user activity based on the correlation of the estimated cognitive ability index with the at least one cognitive health index.

The at least one cognitive health index may indicate at least one of a physical fitness, a mental fitness, and vulnerability of the user to perform an activity.

The change in the at least one cognitive ability of the user may correspond to a change in cognitive health of the user to perform a task comprising at least one of a physical activity and a mental activity.

The determined at least one corrective action may correspond to an action that, when performed by the user, results in a mitigation of the predicted at least one possible physical risk that may occur during the current user activity.

The method may include analyzing the multi-modal input data, wherein the multi-modal input data includes a plurality of parameters related to the at least one multi-modal interaction of the user; classifying each of a corresponding parameter from the plurality of parameters related to the at least one multi-modal interaction of the user; and generating an updated multi-modal input data based on the classification of the corresponding parameter of the plurality of parameters.

The method may include monitoring, over the first time period, at least one user activity of the user in the IoT environment; and generating multi-modal cognitive data corresponding to the user based on the monitored multi-modal input data and the monitored at least one user activity, the multi-modal cognitive data comprising information related to the plurality of cognitive health indexes.

The monitored at least one user activity of the user may be a location-based activity corresponding to different locations within the IoT environment.

The identifying the change in the at least one cognitive ability of the user includes: obtaining predefined cognitive decline criteria from a knowledge database; converting the generated the multi-modal cognitive data into a plurality of weighted standard cognitive indexes based on the obtained predefined cognitive decline criteria; comparing a corresponding cognitive health index of the plurality of cognitive health indexes with a corresponding weighted standard cognitive index among the plurality of weighted standard cognitive indexes; and identifying the change in the at least one cognitive ability of the user based on a result of the comparison.

The determining the at least one corrective action includes: predicting a capability of the user to handle the predicted at least one possible physical risk associated with the current user activity based on the cognitive ability index of the user; predicting a type of the predicted at least one possible physical risk based on the predicted capability of the user to handle the predicted at least one possible physical risk; and determining the at least one corrective action based on the predicted capability of the user to handle the predicted at least one possible physical risk and the predicted type of the at least one possible physical risk.

The method may include predicting a physical location within the IoT environment where the predicted at least one possible physical risk may occur during the current user activity based on a detection of a user's location within the IoT environment and an identification of a type of the current user activity; and controlling at least one IoT device in the IoT environment to notify the determined at least one corrective action based on the predicted physical location to mitigate the predicted at least one possible physical risk.

The multi-modal input data may include information regarding cognitive health parameters associated with the user, and the cognitive health parameters may correspond to at least one of Montreal Cognitive Assessment (MoCA), circadian rhythm disruption computation (CRDC), a Blood alcohol concentration (BAC) value, a percentage of water in user's body, chronic illness including arthritis, vitamin B-12 deficiency, underactive thyroid gland, and diabetic condition, a level of each of Dementia, Alzheimer's, Parkinson's, and cardiovascular diseases, and a level of blood sugar during at least one of pre-meal, post-meal, fasting.

According to an aspect of the disclosure a system for mitigating physical risks associated with a user in an Internet of Things (IoT) environment, includes: a memory storing instructions; and at least one processor configured to execute the instructions to: monitor multi-modal input data over a first time period, the multi-modal input data being associated with at least one multi-modal interaction with at least one IoT device in the IoT environment; identify a change in at least one cognitive ability of the user, based on the monitored multi-modal input data, estimate a cognitive ability index of the user based on the identified change in the at least one cognitive ability of the user; predict at least one possible physical risk associated with a current user activity in the IoT environment based on a correlation of the estimated cognitive ability index with at least one cognitive health index from a plurality of cognitive health indexes, determine at least one corrective action to avoid the predicted at least one possible physical risk associated with the current user activity, and control at least one IoT device in the environment to notify or perform the determined at least one corrective action such that the predicted at least one possible physical risk is mitigated.

The at least one processor may be further configured to execute the instructions to: correlate the estimated cognitive ability index with the at least one cognitive health index; and predict the at least one possible physical risk associated with the current user activity based on the correlation of the estimated cognitive ability index with the at least one cognitive health index.

The at least one processor may be further configured to execute the instructions to: analyze the multi-modal input data, wherein the multi-modal input data includes a plurality of parameters related to the at least one multi-modal interaction of the user; classify each of a corresponding parameter from the plurality of parameters related to the at least one multi-modal interaction of the user, and generate an updated multi-modal input data based on the classification of the corresponding parameter of the plurality of parameters.

The at least one processor is further configured to execute the instructions to: monitor, over the first time period, at least one user activity of the user in the IoT environment, and generate multi-modal cognitive data corresponding to the user based on the monitored multi-modal input data and the monitored at least one user activity, the multi-modal cognitive data comprising information related to the plurality of cognitive health indexes.

The at least one processor may be further configured to execute the instructions to: obtain predefined cognitive decline criteria from a knowledge database, convert the generated the multi-modal cognitive data into a plurality of weighted standard cognitive indexes based on the obtained predefined cognitive decline criteria, compare a corresponding cognitive health index of the plurality of cognitive health indexes with a corresponding weighted standard cognitive index among the plurality of weighted standard cognitive indexes, and identify the change in the at least one cognitive ability of the user based on a result of the comparison.

The at least one processor may be further configured to execute the instructions to: predict a capability of the user to handle the predicted at least one possible physical risk associated with the current user activity based on the cognitive ability index of the user, predict a type of the predicted at least one possible physical risk based on the predicted capability of the user to handle the predicted at least one possible physical risk, and determine the at least one corrective action based on the predicted capability of the user to handle the predicted at least one possible physical risk and the predicted type of the at least one possible physical risk.

The at least one processor is further configured to execute the instructions to: predict a physical location within the IoT environment where the predicted at least one possible physical risk may occur during the current user activity based on a detection of a user's location within the IoT environment and an identification of a type of the current user activity, and control at least one IoT device in the IoT environment to notify the determined at least one corrective action based on the predicted physical location to mitigate the predicted at least one possible physical risk.

According to an aspect of the disclosure, a non-transitory computer readable medium includes computer readable program code or instructions which are executable by a processor to perform a method for mitigating physical risks associated with a user in an Internet of Things (IoT) environment. The method includes: monitoring multi-modal input data over a first time period, the multi-modal input data being associated with at least one multi-modal interaction of the user with at least one IoT device in the IoT environment; identifying a change in at least one cognitive ability of the user, based on the monitored multi-modal input data; estimating a cognitive ability index of the user, based on the identified change in the at least one cognitive ability of the user; predicting at least one possible physical risk associated with a current user activity in the IoT environment based on a correlation of the estimated cognitive ability index with at least one cognitive health index from a plurality of cognitive health indexes; determining at least one corrective action to avoid the predicted at least one possible physical risk associated with the current user activity; and controlling at least one IoT device in the environment to notify or perform the determined at least one corrective action such that the predicted at least one possible physical risk is mitigated.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 is a block diagram of a system architecture illustrating a smart home system, in accordance with an embodiment;

FIG. 2 is a block diagram illustrating a system for mitigating physical risks associated with a user in an IoT environment, in accordance with an embodiment;

FIG. 3 is a flowchart of a method for mitigating physical risks associated with a user in an IoT environment, in accordance with an embodiment;

FIG. 4 is a flowchart of a method for identifying a change in one or more cognitive abilities of a user, in accordance with an embodiment;

FIG. 5 is a flowchart of a method for determining one or more corrective actions of a user, in accordance with an embodiment;

FIG. 6 is a block diagram illustrating an example architecture to provide tools and a development environment for a technical realization of the system of FIG. 2, in accordance with an embodiment; and

FIG. 7 illustrates a typical hardware configuration, in accordance with an embodiment.

### DETAILED DESCRIPTION

It should be understood at the outset that although various embodiments of the present disclosure are illustrated below, the present disclosure may be implemented using any number of techniques, whether currently known or in existence. The present disclosure should in no way be limited to the illustrative implementations, drawings, and techniques illustrated below, including the exemplary design and implementation illustrated and described herein, but may be modified within the scope of the appended claims along with their full scope of equivalents.

Further, skilled artisans will appreciate that elements in the drawings are illustrated for simplicity and may not have necessarily been drawn to scale. For example, the flowcharts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having the benefit of the description herein.

The term “some” as used herein is defined as “none, or one, or more than one, or all.” Accordingly, the terms “none,” “one,” “more than one,” “more than one, but not all” or “all” would all fall under the definition of “some.” The term “some embodiments” may refer to no embodiments or one embodiment or several embodiments or all embodiments. Accordingly, the term “some embodiments” is defined as meaning “no embodiment, or one embodiment, or more than one embodiment, or all embodiments.”

The terminology and structure employed herein are for describing, teaching, and illuminating some embodiments and their specific features and elements and do not limit, restrict, or reduce the spirit and scope of the claims or their equivalents.

More specifically, any terms used herein such as but not limited to “includes,” “comprises,” “has,” “consists,” and grammatical variants thereof do not specify an exact limitation or restriction and certainly do not exclude the possible addition of one or more features or elements, unless otherwise stated, and must not be taken to exclude the possible removal of one or more of the listed features and elements, unless otherwise stated.

Whether or not a certain feature or element was limited to being used only once, either way, it may still be referred to as “one or more features” or “one or more elements” or “at least one feature” or “at least one element.” Furthermore, the use of the terms “one or more” or “at least one” feature or element do not preclude there being none of that feature or element unless otherwise stated.

The term “unit” used in the present disclosure may imply a unit including, for example, one of hardware, software, and firmware or a combination of two or more of them. The “unit” may be interchangeably used with a term such as logic, a logical block, a component, a circuit, and the like. The “unit” may be a minimum system component for performing one or more functions or may be a part thereof. The “unit” may be electrically implemented. For example, the “units” of the present disclosure may include at least one of an Application-Specific Integrated Circuit (ASIC) chip, a Field-Programmable Gate Arrays (FPGAs), and a programmable-logic device, which are known or will be developed, and which perform certain operations.

Unless otherwise defined, all terms, and especially any technical and/or scientific terms, used herein may be taken to have the same meaning as commonly understood by one having ordinary skill in the art.

Various embodiments of the present disclosure will be described below in detail with reference to the accompanying drawings.

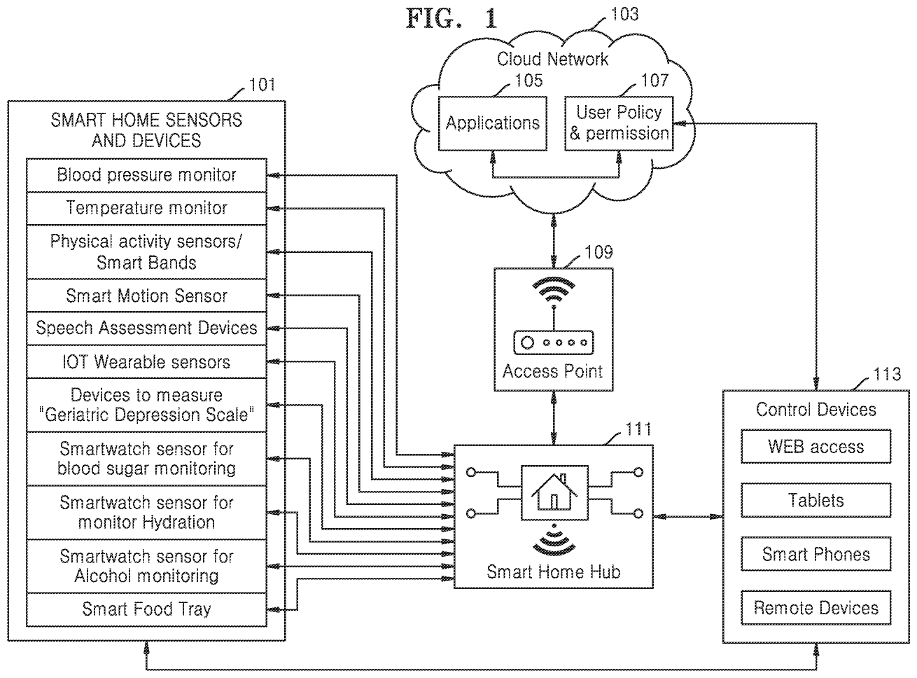

FIG. 1 is a block diagram of a system architecture illustrating a smart home system, in accordance with an embodiment. The smart home system includes smart home sensors and devices 101, cloud network 103 including cloud applications 105 and user policy and permission function 107, an access point 109, a smart home hub 111, and control devices 113. The home sensors and devices 101 may include devices but are not limited to, blood pressure monitor, temperature monitor, physical activity sensors/smart bands, smart motion sensors, speech assessment devices, IoT wearable sensors, and devices to measure “the Geriatric Depression Scale”, smartwatch sensor for blood sugar monitoring, smartwatch sensor to monitor hydration, smartwatch sensor for alcohol monitoring, smart food tray, and other IoT devices and sensors within the scope of the IoT.

The home sensors and devices 101 are IoT-enabled devices and may, for example, measure physical quantities or detect various parameters related to a health condition of a user, and convert the measured or detected parameters into an electrical signal. The home sensors and devices 101 may further include one or more sensors to determine environmental conditions within an area of the smart home system. The one or more sensors may include but are not limited to, IR sensors, humidity sensors, pressure sensors, level sensors, gas sensors, temperature sensors, and the like.

The cloud network 103 may refer to a network that forms part of a cloud computing infrastructure or exists in a cloud computing infrastructure. The cloud network 103 may include one or more cloud servers that may act as the service infrastructure of cloud service providers that intend to provide cloud services to users. The cloud network 103 may manage user policy and permission function 107 with respect to the IoT devices connected to the access point 109. Some of the IoT devices that are connected to the access point 109 may be provided with full access and some of the IoT devices may be provided with a limited access user policy.

The smart home system incorporates a single-platform structure where all the smart devices and sensors are connected to access point 109. The smart home sensors and devices 101 may be connected to each other via the smart home hub 111 through the access point. The access point 109 may control multiple hardware or software components connected to the access point 109 by driving, for example, the operating system (OS) or application programs, and may perform various data processing and calculations. The access point 109 may be implemented as, for example, but is not limited to, a system on chip (SoC). The access point 109 may further include a communication processor to control communication with the cloud network 103.

The smart home hub 111 is configured to control the home sensors and devices 101. The smart home hub 111 corresponds to home-automation controllers and provides a centralized UI that may be used by any user within the smart home at any time. The smart home hub 111 is typically connected to the access point 109 and allows the integration of multiple home automation devices for centralized control of the various home automation devices. Smart hubs typically include a software application interface, often a smartphone application. The smart home hub 111 may also include a virtual smart assistant for voice recognition and voice-controlled automation.

The control devices 113 may be stand-alone devices or may include at least one of a home networking controller implemented as smartphones, tablets, and remote devices, a media device, a network access point as Web access, a control panel, or any combination thereof. The control devices 113 may be wired to and/or wirelessly connected to various external electronic devices (for example, IoT devices), using various communication schemes. For example, the communication schemes may include at least one of, but are not limited to, Wireless Fidelity (WiFi), Bluetooth (BT), Bluetooth Low Energy (BLE), Zigbee, power line communication, Infrared transmission (IR), ultrasound communication, and the like. The control device 113 may be connected to the home sensors and devices 101 and perform a function of controlling home sensors and devices 101 and communicating data with the home sensors and devices 101. In other embodiments, the control devices 113 may serve as a gateway that collects data from the home sensors and devices 101 and forwards the collected data to the cloud network 103 or the smart home hub 111 through the Internet. The cloud network 103 may collect data from the control devices 113 and other devices similar to the control device 113. The collected data may be used for a specific purpose. In certain embodiments, the control devices 113 may also be connected to a personal cloud (for example, DropBox®, iCloud®, SugarSync®, SkyDrive®, OneDrive®, GoogleDrive®, and the like) for collecting and storing the data from the home sensors and devices 101.

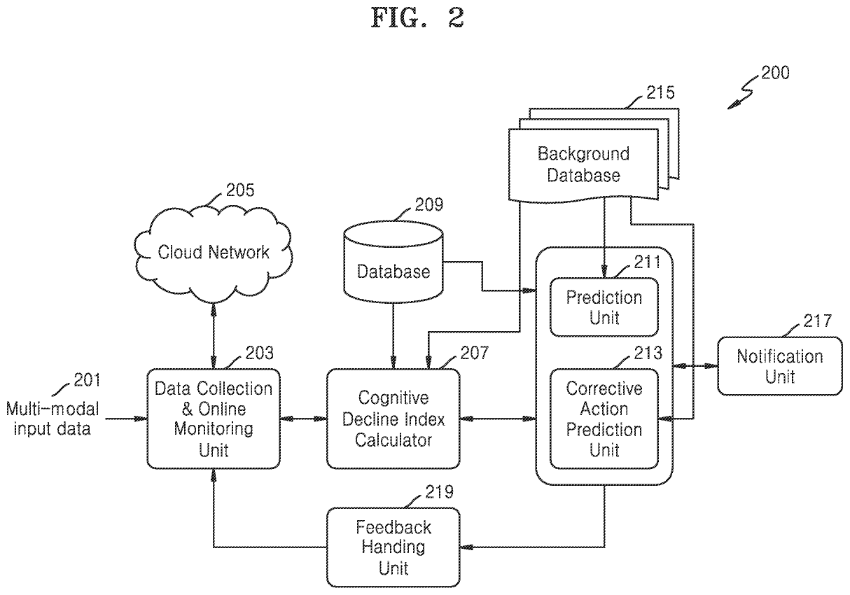

FIG. 2 is a block diagram illustrating a system for mitigating physical risks associated with a user in an IoT environment, in accordance with an embodiment. The system 200 includes a data collection and online monitoring unit 203, a cloud network 205, a cognitive decline index calculator 207, a database 209, a prediction unit 211, a corrective action prediction unit 213, a Background database 215, a notification unit 217, and a feedback handling unit 219. Here, cloud network 205 is the same as cloud network 103 of FIG. 1, and therefore a description of the functionality of cloud network 205 will be omitted herein for the sake of brevity.

The aforementioned components of the system 200 are connected with each other wired/wirelessly for transfer of data from one component to the other component within the system 200. It will be understood to a person of ordinary skill in the art that the present disclosure is not limited to the system architecture of FIG. 2, the concept of the proposed architecture can be applied to any kind of IoT system to be implemented in near future for mitigating accidents or physical risks associated in the IoT environment.

The data collection and online monitoring unit 203 is configured to perform personalized monitoring activities and generates log data including the result of the monitoring process. In particular, the data collection and online monitoring unit 203 monitors over a time period one or more user activities of the user in the IoT. The monitored one or more user activities of the user corresponds to location-based activities corresponding to different locations within the IoT environment (For example, location including, but are not limited to, kitchen, bathroom, living room, bedroom, balcony, or any other indoor home area). The data collection and online monitoring unit 203 may correspond to smart devices and monitoring devices that perform monitoring using one or more sensors, and a camera with an image sensor. The image sensor corresponds to one or more optical sensors and may include a charge-coupled device (CCD) or complementary metal-oxide-semiconductor (CMOS) phototransistors. The one or more optical sensors receive light from the environment, are projected through one or more lenses, and convert the light to data representing an object or subject image. The one or more optical sensors may capture images or video. In order to capture one or more images, the data collection and online monitoring unit 203 may include a camera assembly including one or more image sensors, or pair of cameras provided as part of a single element.

The data collection and online monitoring unit 203 is configured to monitor over a time period multi-modal input data associated with one or more multi-modal interactions with one or more devices among the home sensors and devices 101 in the IoT environment. The multi-modal input data is data that is collected by the data collection and online monitoring unit 203 using the home sensors and devices 101, other Smart Devices, and IoT Wearable sensors within the IoT environment. The multi-modal input data includes a plurality of parameters related to the one or more multi-modal interactions of the user. The multi-modal interactions are interactions that are performed by the user with real smart home devices via text input, speech input, or the like. The multi-modal interaction may also involve interaction technique such as eye blinking or touch-based interactions that allows the user with limited physical mobility to control the home sensors and devices 101 in the IoT environment (smart home system). The data collection and online monitoring unit 203 may also be configured to analyze a result of the monitoring of the multi-modal input data, classify each of a corresponding parameter of the plurality of parameters related to the one or more multi-modal interactions of the user, and thereafter generate an updated (new) multi-modal input data based on the classification. The data collection and online monitoring unit 203 is further configured to generate multi-modal cognitive data corresponding to the user based on the monitored multi-modal input data and the monitored one or more user activities. The multi-modal cognitive data generated by the data collection and online monitoring unit 203 includes information related to the cognitive health indexes of the user.

The cognitive decline index calculator 207 is configured to identify a change in one or more cognitive abilities of the user by applying a Machine Learning (ML) model on an output of the data collection and online monitoring unit 203. Here, the change in the one or more cognitive abilities of the user corresponds to a change in cognitive health of the user for performing a task including physical activity and mental activity within the IoT environment. The change in cognitive health of the user can be referred to as a change in the health state or health condition of the user for performing the tasks. The output of the data collection and online monitoring unit 203 corresponds to a result of the monitoring of the multi-modal input data. In accordance with an embodiment of the present disclosure, the cognitive decline index calculator 207 is further configured to estimate a cognitive ability index of the user based on the identified change in the one or more cognitive abilities of the user.

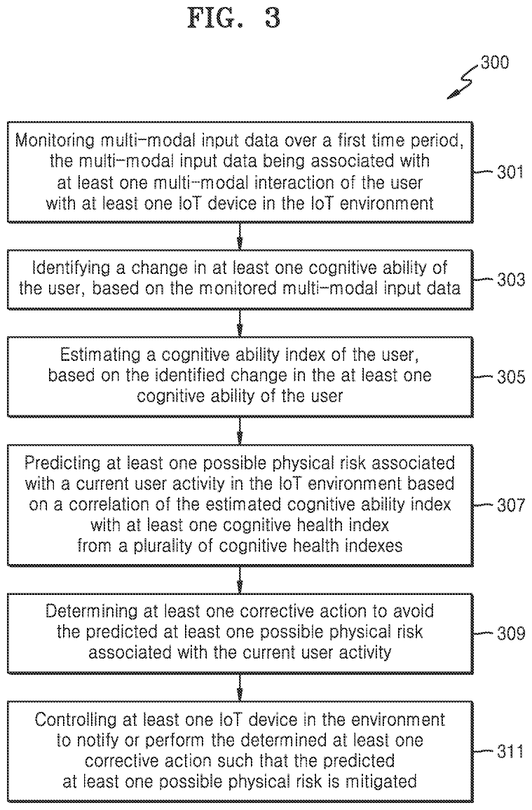

Further, the functionalities of the prediction unit 211, the corrective action prediction unit 213, the notification unit 217, and the feedback handling unit 219 will be explained with reference to the flowcharts illustrated in FIGS. 3, 4, and 5.

FIG. 3 illustrates a flowchart of a method for mitigating physical risks associated with a user in an IoT environment, in accordance with an embodiment. FIG. 3 depicts a method 300 that is executed by components of the system 200 of FIG. 2. The detailed description of the functionalities of the components of the system 200 will now be made with respect to the flow of method 300 with reference to FIGS. 3-5.

The method 300 as depicted in FIG. 3, at 301, comprises monitoring the multi-modal input data associated with the one or more multi-modal interactions of the user with one or more IoT devices present in the IoT environment. As an example, the data collection and online monitoring unit 203 monitors the one or more multi-modal interactions of the user with the home sensors and devices 101 present in the IoT environment. At first, the data collection and online monitoring unit 203 collects the multi-modal input data using at least one of the blood pressure monitor, temperature monitor, physical activity sensors/smart bands, smart motion sensor, speech assessment devices, IoT wearable sensors, devices to measure “Geriatric Depression Scale”, smartwatch sensor for blood sugar monitoring, smartwatch sensor to monitor hydration, smartwatch sensor for alcohol monitoring, smart food tray, and other IoT devices within the IoT environment. Thereafter, the data collection and online monitoring unit 203 organizes and normalize the collected multi-modal input data for computation of one or more cognitive decline index.

The collected multi-modal input data includes information regarding cognitive health parameters associated with the user. For example, the cognitive health parameters correspond to parameters including, but are not limited to, Montreal Cognitive Assessment (MoCA), circadian rhythm disruption computation (CRDC), a blood alcohol concentration (BAC) value, a percentage of water in the user's body for well-functioning, chronic illness including arthritis, vitamin B-12 deficiency, underactive thyroid gland, and diabetic condition, a level of each of Dementia, Alzheimer's, Parkinson's, and cardiovascular diseases, and a level of blood sugar during at least one of pre-meal, post-meal, fasting. The health parameters may also include an estimate of the change in food component intake percentage based on each periodic serving. The MoCA measure indicates values for cognitive impairment in a speech of the user. The CRDC measures indicate levels of sleep characteristics including sleep duration, sleep efficiency, sleep latency, and sleep fragmentation.

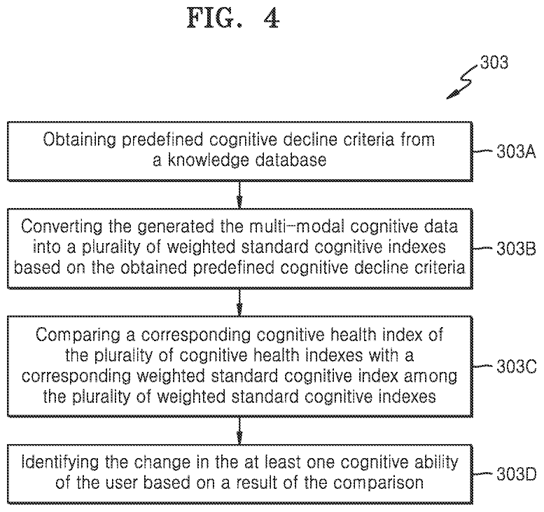

At 303, subsequent to the monitoring of the one or more multi-modal interactions of the user with one or more IoT devices, the method 300 comprises identifying, based on the monitored multi-modal input data, the change in the one or more cognitive abilities of the user for performing the physical tasks or mental tasks. As an example, the identification of the change in the one or more cognitive abilities of the user will be explained in accordance with the flowchart illustrated in FIG. 4.

FIG. 4 is a flowchart of a method for identifying a change in one or more cognitive abilities of a user, in accordance with an embodiment.

At 303A, the cognitive decline index calculator 207 obtains predefined cognitive decline criteria from a knowledge database (e.g., database 209). The database 209 is a relational database wherein various database tables having standard medical research information are employed as basis for cognitive decline rules for determining the one or more cognitive abilities of the user. At 303B, the cognitive decline index calculator 207 converts the multi-modal cognitive data generated by the data collection and online monitoring unit 203 into weighted standard cognitive indexes based on the obtained predefined cognitive decline criteria (e.g., medically established criteria for determining standard cognitive indexes) and background metadata stored in the background database 215. The metadata includes predefined cognitive decline information related to the user and associated cognitive decline labels for accidents and the inability of users to do the self-support. The cognitive decline labels contain but are not limited to the list of the cognitive decline components which may be responsible for accidents and the inability of the user to do the self-support (to overcome, if accidents happen). The list may include various cognitive decline components including, but are not limited to cognitive impairment of speech, Levels of (a) Dementia, (b) Alzheimer's, (c) Parkinson's, and (d) CVD, capacity to hold objects by hand, control over body and balance, ability to organize tasks, emotional imbalance/depression, hydration status of the body, presence of alcohol content in the body, food intake decline, sleep disorder caused cognitive decline, short-term/long-term memory loss, and like. Each of these cognitive decline components can be classified into one or more class labels including, but is not limited to, “High”, “Moderate”, or “Low”.

The weighted standard cognitive indexes indicate different types of personalized cognitive decline indexes of the user for performing the physical or the mental tasks. For example. a standard cognitive index of MoCA having a weightage score greater than or equal to 14 indicates mild cognitive impairment of speech, and a standard cognitive index of MoCA having a weightage score greater than or equal to 18 indicates saviour cognitive impairment. In another example, the depression levels of the user may depend on the user's age, user's education, and complaints. The severity level of depression having a weightage score in a range of 5-8 indicates a mild depression level, the severity level of depression having a weightage score in a range of 9-11 indicates moderate depression level, and the severity level of depression having a weightage score in a range of 12-15 indicates severe depression level. Those skilled in the art will appreciate that the aforementioned examples of weightage score are merely exemplary and is not intended to limit the scope of the disclosure.

At 303C, the cognitive decline index calculator 207 compares a corresponding cognitive health index of the cognitive health indexes with a corresponding weighted standard cognitive index among the weighted standard cognitive indexes. As an example, a weightage score for a cognitive index of MoCA is calculated as 17 in real-time, and a standard cognitive index of MoCA is equal to 14. When these both scores are compared with each other, a result of the comparison indicates a difference of percentage in the score e.g., +03% (percentage change in the cognitive index). Those skilled in the art will appreciate that the aforementioned example of calculating the difference in the weightage score is merely exemplary and is not intended to limit the scope of the disclosure.

At 303D, the cognitive decline index calculator 207 identifies the change in the one or more cognitive abilities of the user based on the result of the comparison between the corresponding cognitive health index of the cognitive health indexes with the corresponding weighted standard cognitive index among the weighted standard cognitive indexes. An example scenario representing the change in the one or more cognitive abilities of a plurality of users (e.g., user with ID 1, ID 2, and ID 3) are shown below in Tables 1, 2, and 3 respectively based on the comparison between their corresponding cognitive health index with the corresponding weighted standard cognitive index. A negative change (−) change in the cognitive abilities indicates a worsening of the situation, while a positive (+) change in the cognitive abilities indicates improvement in the situation. Those skilled in the art will appreciate that the below-mentioned information in the example Tables 1, 2, and 3 are merely exemplary and is not intended to limit the scope of the disclosure. Tables 1, 2, and 3 may include a different set of information based on other cognitive decline parameters or components.

TABLE 1

| Cognitive Decline Indexes which are | | |
|---|---|---|
| above the safety limit (others are not | Weight in % or | % |
| discussed here) for person having ID #1 | Standard Units | Change |
| 1. MoCA - Measure for Speech | 17.00 | +05 |
| 2. Depression level | 11.50 | +01 |
| 3. Capacity to hold objects by Hand | 80.00% | −04 |
| 4. Control Over Body and Balance | 80.00% | −05 |
| 5. Ability to Organize tasks | 70.00% | +10 |
| 6. Emotional imbalance | 55.00% | −10 |
| 7. Blood Sugar (in mg/dL) [a, b, c, d] | [160, 200, 140, 220] | −30 |
| 8. BAC | [70] | +01 |
| 9. CRDC | [04, 25%, 01, 11] | −20 |
| 10. Alzheimer Stage | Stage-3 | +00 |
| 11. Blood Pressure | [90/65] | −10 |

TABLE 2

| Cognitive Decline Indexes which are | | |
|---|---|---|
| above the safety limit (others are not | Weight in % or | % |
| discussed here) for person having ID #2 | Standard Units | Change |
| 1. MoCA - Measure for Speech | 17.00 | +05 |
| 2. Depression level | 11.50 | +01 |
| 3. Capacity to hold objects by Hand | 80.00% | −04 |
| 4. Control Over Body and Balance | 80.00% | −05 |
| 5. Blood Pressure | [50/75] | −30 |
| 6. Emotional imbalance | 55.00% | −10 |
| 7. Blood Sugar (in mg/dL) [a, b, c, d] | [160, 200, 140, 220] | −30 |
| 8. BAC | [70] | +01 |
| 9. CRDC | [04, 25%, 01, 11] | −20 |
| 10. Alzheimer Stage | Stage-3 | +00 |

TABLE 3

| Cognitive Decline Indexes which are | | |
|---|---|---|
| above the safety limit (others are not | Weight in % or | % |
| discussed here) for person having id #3 | Standard Units | Change |
| 1. Cognitive impairment of speech | 60.00% | +05 |
| 2. Increase level of Dementia | 71.00% | +01 |
| 3. Capacity to hold objects by Hand | 70.00% | −20 |
| 4. Control Over Body and Balance | 75.00% | −20 |
| 5. Ability to Organize tasks | 60.00% | −10 |
| 6. Emotional imbalance | 55.00% | −10 |
| 7. Blood Sugar (in mg/dL) [a, b, c, d] | [160, 200, 140, 220] | −30 |
| 8. BAC | [110] | +30 |
| 9. CRDC | [05, 30%, 01, 11] | −10 |

At 305, subsequent to the identification of the change in the one or more cognitive abilities of the user, the method 300 comprises estimating a cognitive ability index of the user based on the identified change in the one or more cognitive abilities of the user. As an example, the cognitive decline index calculator 207 estimates the cognitive ability index of the user based on the percentage change in the one or more cognitive abilities of the user for performing the physical and the mental tasks. After the cognitive ability index of the user is estimated, the data along with other information including but is not limited to the physical location (for example bathroom, leaving room, workplace, kitchen, and any other area within the smart home environment), nature of work the user is going to do, time/possible tentative duration for the work, and the like is transferred to the prediction unit 211 and the corrective action prediction unit 213. The cognitive decline features and the parameters that have a negative percentage of change indicates consideration of serious observations.

At 307, subsequent to the estimation of the cognitive ability index of the user, the method 300 comprises predicting one or more possible physical risks associated with a current user activity in the IoT environment based on a correlation of the estimated cognitive ability index of the user with one or more cognitive health index of the user. As an example, the prediction unit 211 predicts the one or more possible accidents associated with the current user activity that may happen in the IoT environment by correlating the estimated cognitive ability index of the user with the one or more cognitive health index of the user. In particular, the different combination of high and/or moderate risk levels of the cognitive decline components or parameters generally results in the different types of accidents. The severity of such accidents increases, if the user finds himself/herself unable to do self-support (in case he or she meets with an accident). Therefore, it becomes very important to predict the possibility of such physical accident's severity levels. Hence, by correlating the estimated cognitive ability index of the user with one or more cognitive health index of the user that is included in the data received by the prediction unit 211 from the cognitive decline index calculator 207, the one or more possible physical risk associated with the current user activity can be predicted using various deep learning models.

The prediction unit 211 may include a deep learning-based processor implemented with deep learning models to train a convolutional neural network (CNN) based on the received data from the cognitive decline index calculator 207. For example, the learning processor may train the neural network to predict results for each of the multiple correlations between the estimated cognitive ability index of the user with the corresponding one or more cognitive health index of the user. The learning processor may likewise train the CNN to learn the features of the data received by the prediction unit 211 from the cognitive decline index calculator 207. The learned features may be referred to as features that may be learned or created in a hidden layer of the CNN. The learned features may indicate how the CNN made the prediction. The prediction unit 211 may perform the prediction to predict the one or more possible physical risks associated with the current user activity by applying the learned features to each of the multiple combinations of information included in the received data. The deep learning-based processor of the prediction unit 211 may also update associated trained modules based on an amount of change in the corresponding cognitive health indexes of the user.

As an example, the prediction unit 211 at first collects ail the data as per requirements of the cognitive decline index calculator 207 and continuously monitors the activities that are directly related to sudden/dynamic changes in health status, personalized and daily activities for better experiences. Then the prediction unit 211, using the machine learning models, predicts the one or more possible physical risks associated with the activities being monitored based on the correlation of the estimated cognitive ability index with one or more cognitive health index of the user indicated by the data collected from the cognitive decline index calculator 207. The one or more cognitive health index of the user calculated by the cognitive decline index calculator 207 indicates at least one of physical fitness, a mental fitness, and vulnerability of the user to perform the physical or the mental activities.

An exemplary example of data to predict the possibility of the one or more possible physical risks/accidents associated with the activities being monitored is shown below in Tables 4, 5, and 6 with respect to the users with the user IDs 1, 2, and 3 respectively. Those skilled in the art will appreciate that the below-mentioned information in the example Tables 4, 5, and 6 are merely exemplary and is not intended to limit the scope of the disclosure. The Tables 4, 5, and 6 may include a different set of information based on any other cognitive decline parameters or components.

TABLE 4

| Predicted high risk of Accident and inability to | |
|---|---|
| do the self support with the following Cognitive | Weight in % for |
| Decline labels | user ID #1 |
| 1. Cognitive impairment of speech | 80.00% |
| 2. Increase level of Dementia | 67.00% |
| 3. Capacity to hold objects by Hand | 70.00% |
| 4. Control Over Body and Balance | 45.00% |
| 5. Ability to Organize tasks | 50.00% |
| 6. Emotional imbalance | 45.00% |

TABLE 5

| Predicted high risk of Accident and inability to | |
|---|---|
| do the self support with the following Cognitive | Weight in % for |
| Decline labels | user ID #2 |
| 1. Cognitive impairment of speech | 80.00% |
| 2. Increase level of Dementia | 67.00% |
| 3. Capacity to hold objects by Hand | 80.00% |
| 4. Control Over Body and Balance | 45.00% |
| 5. Ability to Organize tasks | 50.00% |
| 6. Emotional imbalance | 45.00% |

TABLE 6

| Predicted high risk of Accident and inability to | |
|---|---|
| do the self support with the following Cognitive | Weight in % for |
| Decline labels | user ID #3 |
| 1. Cognitive impairment of speech | 80.00% |
| 2. Increase level of Dementia | 67.00% |
| 3. Capacity to hold objects by Hand | 80.00% |
| 4. Control Over Body and Balance | 45.00% |
| 5. Ability to Organize tasks | 50.00% |
| 6. Emotional imbalance | 45.00% |

At 309, subsequent to the prediction of the one or more possible physical risks associated with the user activity being monitored, the method 300 comprises determining one or more corrective actions to avoid the one or more predicted possible physical risks associated with the user activity being monitored. As an example, the corrective action prediction unit 213 determines, using a multi-tasking self-supervised learning model, the one or more corrective actions in response to the predicted possible physical risks/accidents such that the one or more predicted possible physical risks/accidents associated with the current user activity can be avoided to ensure the user safety. The one or more determined corrective actions correspond to actions that when performed by the user, result in a mitigation of the one or more predicted possible physical risk/accidents that may occur during the current user activity. The one or more corrective actions may include, but are not limited to, a set of actions to avoid/reduce the chances of accidents, necessary warnings, and supporting actions to help the end-user if unfortunately, he/she met with an accident. The corrective action prediction unit 213 may further use the Metadata stored in the background database 215, cognitive decline indexes and cognitive health conditions calculated by the cognitive decline Index calculator 207, accident types, and location information together to determine all kinds of corrective actions for the user.

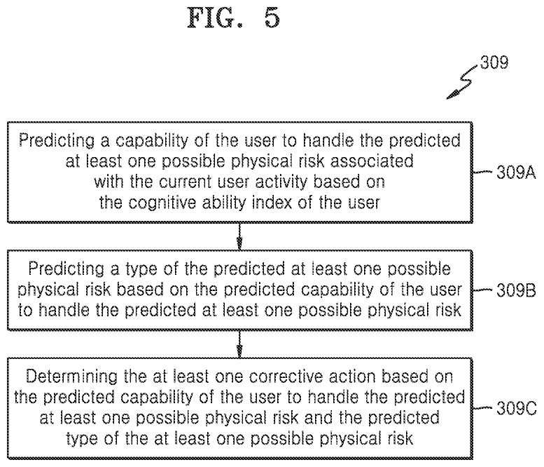

The determination of the one or more corrective actions of the user will be explained in accordance with the flowchart illustrated in FIG. 5.

FIG. 5 is a flowchart of a method for determining one or more corrective actions for the user, in accordance with an embodiment.

At 309A, the corrective action prediction unit 213 predicts a capability of the user to handle the one or more predicted possible physical risks/accidents associated with the user activity based on the cognitive ability index of the user that is estimated at 305 of FIG. 3. At 309B, the corrective action prediction unit 213 predicts a type of the one or more predicted possible physical risks/accidents based on the predicted capability of the user to handle the one or more predicted possible physical risks/accidents. The predicted type of accidents may include but are not limited to, slipping, fall-down, loose control in the bathroom, kitchen, living room, or in any other area within the smart home environment. The predicted type of accident may also include the situation where the user may not be able to do self-support or may not be able to perform any corrective action to minimize the loss. In particular, the different combinations of high and/or moderate risk levels of the cognitive decline components generally result in the different types of accidents. The severity of such accidents increases, if the user finds himself/herself unable to do self-support (in case if he or she meets with an accident). So, it becomes very important to have prior information about such physical accident's severity levels and the corresponding corrective actions. For example, the combination of cognitive decline parameters such as cognitive impairment of speech, levels of (a) Dementia, (b) Alzheimer's, (c) Parkinson's, and (d) CVD, capacity to hold objects by Hand, control over body and balance, and emotional imbalance/depression (with high severity class), may result in slipping, fall-down, loose control type accidents in the bathroom, kitchen, living-room, or in another area within the smart home environment. Due to such a situation, the user may not be able to do self-support or may not be able to perform any corrective action to minimize the loss.

At 309C, the corrective action prediction unit 213 determines the one or more corrective actions for the user based on the predicted capability of the user to handle one or more predicted possible physical risks/accidents and the predicted type of the one or more possible physical risks/accidents. The one or more corrective actions may include, but are not limited to, warning/alerting the user, increasing the light in a working area inside the smart home environment, may use any sound source or smart assistant to check the wellness of the user on a regular interval basis, emergency wakeup of smart devices and smart assistant (with contextual information) and helping the user by making a call to the nearest health center, and the like. The one or more corrective actions may further include personalized corrective actions including, but are not limited to, reminding the user to take medicine on time and the right quantity of medicine, automatic sensing of health decline progress, and warning the user to take precautionary measure, suggest the user to go for a health check, or make a call for the health support, and intelligent reminders to prevent the status of worsening the situation. Furthermore, the one or more corrective actions may include attention based reminders including but are not limited to warning/Alert the user while using harmful or dangerous household equipment, applying safety protocols, and increasing the light and ventilation in working areas within the smart home environment, emergency wakeup of smart assistants with contextual information and help the user by making the call at the nearest health center, fire station, police station, and the like in case of emergency. Such type of emergency wakeups is important in automatically reducing the chances of accidents, and providing appropriate help and support in the case of accidents (without asking so many questions to the user). Those skilled in the art will appreciate that the aforementioned examples of the one or more determined corrective actions are merely exemplary and are not intended to limit the scope of the disclosure.

At 311, subsequent to the determination of the one or more corrective actions for the user, the method 300 comprises controlling one or more IoT devices (e.g., home sensors and devices 101) to notify at least one determined corrective action to the user or to perform at least one determined corrective action such that the one or more predicted possible physical risks/accidents can be mitigated. In particular, as an example, the corrective action prediction unit 213 detects a user's location within the IoT environment or the smart home environment and identifies a type of the current user activity. Thereafter, the corrective action prediction unit 213 predicts at least one physical location within the smart home environment or the IoT environment where the one or more predicted possible physical risks/accidents may occur while the user is performing any activity (e.g., physical activity or mental activity) based on the detected user's location and the identified type of the current user activity. Further, after the prediction of the at least one physical location, the corrective action prediction unit 213 controls, using the notification unit 217, at least one device among the home sensors and devices 101 to notify or perform the one or more determined corrective actions based on the predicted physical location such that the one or more possible physical risks/accidents can be mitigated.

By notifying the corrective actions to the user, the system 200 generally prevents or avoids any future risks/accidents that may occur in the smart home environment. By providing intelligent reminders as the corrective actions may help the user prevents or avoid any future risks/accidents in future. For example, reminders in the case the system detected an irregularity in medicine doses, after the occasional increase or decrease in blood sugar, blood pressure, depression, etc., and risk management in the case of any miss-happening may help the user in preventing bad health conditions that may occur in future. Further, using reminders in the case of forgetting keys, mobile, or other necessary objects while going out for some work due to some cognitive impairment, will help the user to arrange these things beforehand and due to which the possible risks that are to be occurred in near future can be avoided. Further, by providing the user attention-based reminders as the corrective actions may help the user in preventing dangerous accidents or risks that may happen due to lack of attention.

By performing the corrective actions such as, but not limited to making the call for the health support, applying the safety protocols, increasing the light and ventilation in the working areas within the smart home environment, the emergency wakeup of smart assistants with the contextual information and help the user by making the call at the nearest, fire station, police station, and the like in case of emergency, the system 200 generally helps in mitigating the physical risks that may help the user in a situation when the user cannot help himself in case when mat with any physical accidents and hence may avoid any future risks/accidents that may occur in the smart home environment.

Further, in accordance with an embodiment of the present disclosure, the system 200 is provided with feedback handling unit 219 which is configured to feed the user feedback and users inputs collected by the collection and online monitoring unit 203 to the corrective action prediction unit 213 and the prediction unit 211. This feedback data is collected by the prediction unit 211 and the corrective action prediction unit 213 as new metadata and these units utilize the collected feedback data to improve the quality of the prediction of the possible physical risks/accidents and the one or more corrective actions for the user. The prediction unit 211 and the corrective action prediction unit 213 store the feedback data in the Background database 215 every time there is new feedback data is collected and train the deep learning model accordingly to process the new metadata.

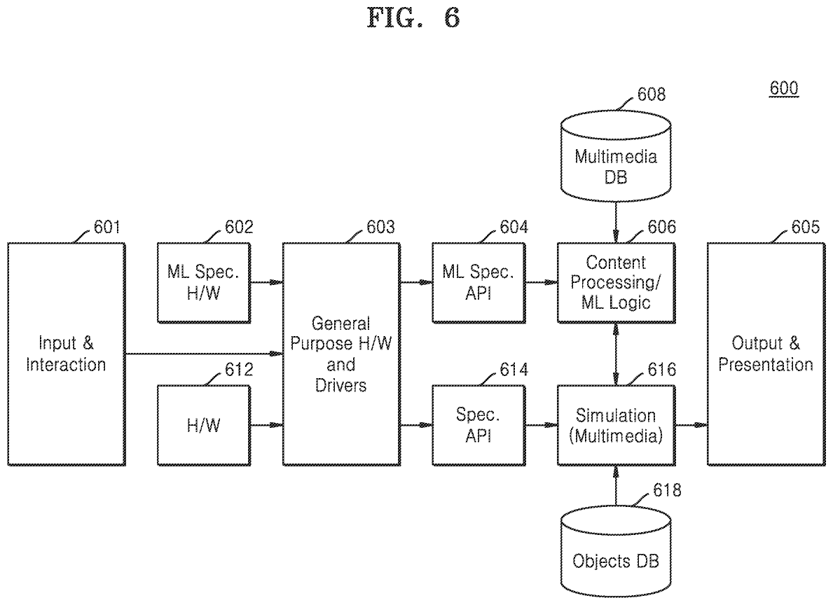

FIG. 6 illustrates an example architecture to provide tools and a development environment described herein for a technical realization of the system 200, in accordance with an embodiment. FIG. 6 is merely a non-limiting example, and it will be appreciated that many other architectures may be implemented to facilitate the functionality described herein. The architecture may be executing on hardware such as a computing machine 600 of FIG. 6 that includes, among other things, processors, memory, and various application-specific hardware components.

The architecture 600 may include an operating system, libraries, frameworks, or middleware. The operating system may manage hardware resources and provide common services. The operating system may include, for example, a kernel, services, and drivers defining a hardware interface layer. The drivers may be responsible for controlling or interfacing with the underlying hardware. For instance, the drivers may include display drivers, camera drivers, Bluetooth® drivers, flash memory drivers, serial communication drivers (e.g., Universal Serial Bus (USB) drivers), Wi-Fi® drivers, audio drivers, power management drivers, and so forth depending on the hardware configuration.

A hardware interface layer includes libraries which may include system libraries such as file-system (e.g., C standard library) that may provide functions such as memory allocation functions, string manipulation functions, mathematic functions, and the like. In addition, the libraries may include API libraries such as audio-visual media libraries (e.g., multimedia data libraries to support presentation and manipulation of various media formats such as MPEG4, H.264, MP3, AAC, AMR, JPG, PNG), database libraries (e.g., SQLite that may provide various relational database functions), web libraries (e.g. WebKit that may provide web browsing functionality), and the like.

A middleware may provide a higher-level common infrastructure such as various graphic user interface (GUI) functions, high-level resource management, high-level location services, and so forth. The middleware may provide a broad spectrum of other APIs that may be utilized by the applications or other software components/modules, some of which may be specific to a particular operating system or platform.

The term “module” or “unit” used in this disclosure may refer to a certain unit that includes one of hardware, software, firmware, or any combination thereof. The module may be interchangeably used with unit, logic, logical block, component, or circuit, for example. The module may be the minimum unit, or part thereof, which performs one or more particular functions. The module may be formed mechanically or electronically. For example, the module or the unit disclosed herein may include at least one ASIC (Application-Specific Integrated Circuit) chip, FPGAs (Field-Programmable Gate Arrays), and programmable-logic device, which have been known or are to be developed.

Further, the architecture 600 depicts an aggregation of audio/video processing device-based mechanisms and ML/NLP-based mechanisms in accordance with an embodiment of the present subject matter. A user interface is defined as input and interaction 601 refers to overall input. It can include one or more of the following—touch screen, microphone, camera, etc. A first hardware module 602 depicts specialized hardware for ML/NLP based mechanisms. As an example, the first hardware module 602 comprises one or more neural processors, FPGA, DSP, GPU, etc.

A second hardware module 612 depicts specialized hardware for executing the data splitting and transfer. ML/NLP based frameworks and APIs 604 correspond to the hardware interface layer for executing the ML/NLP logic 606 based on the underlying hardware. In an example, the frameworks may be one or more of the following—Tensorflow, Café, NLTK, GenSim, ARM Compute, etc. Simulation frameworks and APIs 614 may include one or more of—Audio Core, Audio Kit, Unity, Unreal etc.

A database 608 depicts a pre-trained database. The database 608 may be remotely accessible through cloud by the ML/NLP logic 606. In other example, the database 608 may partly reside on cloud and partly on-device based on usage statistics.

Another database 618 refers the memory. The database 618 may be remotely accessible through cloud. In other example, the database 618 may partly reside on the cloud and partly on-device based on usage statistics.

A rendering module 605 is provided for rendering audio output and triggering further utility operations. The rendering module 605 may be manifested as a display cum touch screen, monitor, speaker, projection screen, etc.

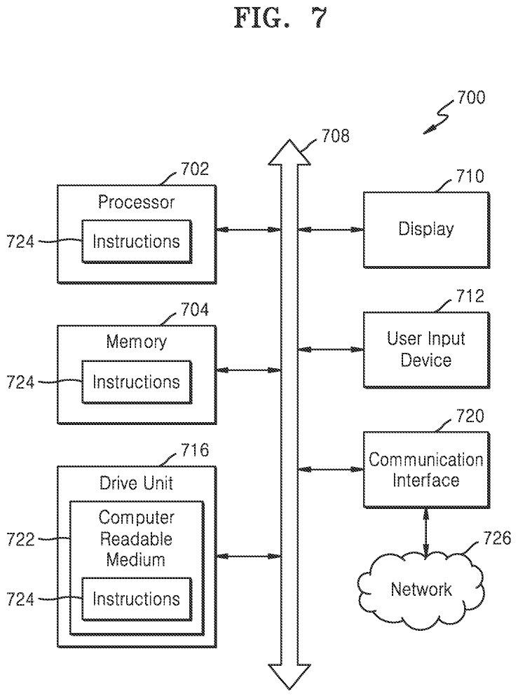

A general-purpose hardware and driver module 603 corresponds to the computer system 700 as referred in FIG. 7 and instantiates drivers for the general-purpose hardware units as well as the application-specific units (602, 612).

In an example, the ML mechanism underlying the present architecture 600 may be remotely accessible and cloud-based, thereby being remotely accessible through a network connection. An audio/video processing device may be configured for remotely accessing the NLP/ML modules and simulation modules may comprise skeleton elements such as a microphone, a camera a screen/monitor, a speaker, etc.

Further, at least one of the plurality of modules of the mesh network may be implemented through AI based on an ML/NLP logic 606. A function associated with AI may be performed through the non-volatile memory, the volatile memory, and the processor constituting the first hardware module 602 e.g., specialized hardware for ML/NLP based mechanisms. The processor may include one or a plurality of processors. At this time, one or a plurality of processors may be a general-purpose processor, such as a central processing unit (CPU), an application processor (AP), or the like, a graphics-only processing unit such as a graphics processing unit (GPU), a visual processing unit (VPU), and/or an AI-dedicated processor such as a neural processing unit (NPU). The aforesaid processors collectively correspond to the processor 702 of FIG. 7.

One or a plurality of processors control the processing of the input data in accordance with a predefined operating rule or artificial intelligence (AI) model stored in the non-volatile memory and the volatile memory. The predefined operating rule or artificial intelligence model is provided through training or learning.

Here, being provided through learning means that, by applying a learning logic/technique to a plurality of learning data, a predefined operating rule or AI model of the desired characteristic is made. “Obtained by training” means that a predefined operation rule or artificial intelligence model configured to perform the desired feature (or purpose) is obtained by training a basic artificial intelligence model with multiple pieces of training data by a training technique. The learning may be performed in a device (e.g., the architecture 600 or the system 700) itself in which AI according to an embodiment is performed, and/or may be implemented through a separate server/system. “

The AI model may consist of a plurality of neural network layers. Each layer has a plurality of weight values and performs a neural network layer operation through calculation between a result of computation of a previous layer and an operation of a plurality of weights. Examples of neural networks include, but are not limited to, convolutional neural network (CNN), deep neural network (DNN), recurrent neural network (RNN), restricted Boltzmann Machine (RBM), deep belief network (DBN), bidirectional recurrent deep neural network (BRDNN), generative adversarial networks (GAN), and deep Q-networks.

The ML/NLP logic 606 is a method for training a predetermined target device (for example, a robot) using a plurality of learning data to cause, allow, or control the target device to make a determination or prediction. Examples of learning techniques include, but are not limited to, supervised learning, unsupervised learning, semi-supervised learning, or reinforcement learning.

FIG. 7 illustrates a typical hardware configuration of the system 600 in the form of a computer system 700, in accordance with an embodiment. The computer system 700 can include a set of instructions that can be executed to cause the computer system 700 to perform any one or more of the methods disclosed. The computer system 700 may operate as a standalone device or may be connected, e.g., using a network, to other computer systems or peripheral devices.

In a networked deployment, the computer system 700 may operate in the capacity of a server or as a client user computer in a server-client user network environment, or as a peer computer system in a peer-to-peer (or distributed) network environment. The computer system 700 can also be implemented as or incorporated across various devices, such as a personal computer (PC), a tablet PC, a personal digital assistant (PDA), a mobile device, a palmtop computer, a laptop computer, a desktop computer, a communications device, a wireless telephone, a land-line telephone, a web appliance, a network router, switch or bridge, or any other machine capable of executing a set of instructions (sequential or otherwise) that specify actions to be taken by that machine. Further, while the computer system 700 is illustrated as a single computer system, the term “system” shall also be taken to include any collection of systems or sub-systems that individually or jointly execute a set, or multiple sets, of instructions to perform one or more computer functions.

The computer system 700 may include a processor 702 e.g., a central processing unit (CPU), a graphics processing unit (GPU), or both. The processor 702 may be a component in a variety of systems. For example, the processor 702 may be part of a standard personal computer or a workstation. The processor 702 may be one or more general processors, digital signal processors, application-specific integrated circuits, field-programmable gate arrays, servers, networks, digital circuits, analog circuits, combinations thereof, or other now known or later developed devices for analyzing and processing data. The processor 702 may implement a software program, such as code generated manually (e.g., programmed).

The computer system 700 may include a memory 704, such as a memory 704 that can communicate via a bus 708. The memory 704 may include but is not limited to computer-readable storage media such as various types of non-transitory volatile and non-volatile storage media, including but not limited to random access memory, read-only memory, programmable read-only memory, electrically programmable read-only memory, electrically erasable read-only memory, flash memory, magnetic tape or disk, optical media and the like. In one example, memory 704 includes a cache or random access memory for the processor 702. In alternative examples, the memory 704 is separate from the processor 702, such as a cache memory of a processor, the system memory, or other memory. The memory 704 may be an external storage device or database for storing data. The memory 704 is operable to store instructions executable by the processor 702. The functions, acts or tasks illustrated in the figures or described may be performed by the processor 702 programmed for executing the instructions stored in the memory 704. The functions, acts or tasks are independent of the particular type of instructions set, storage media, processor or processing strategy and may be performed by software, hardware, integrated circuits, firmware, micro-code and the like, operating alone or in combination. Likewise, processing strategies may include multiprocessing, multitasking, parallel processing and the like.

As shown, the computer system 700 may or may not further include a display unit 710, such as a liquid crystal display (LCD), an organic light-emitting diode (OLED), a flat panel display, a solid-state display, a projector, a printer or other now known or later developed display device for outputting determined information. The display unit 710 may act as an interface for the user to see the functioning of the processor 702, or specifically as an interface with the software stored in the memory 704 or the drive unit 716.

Additionally, the computer system 700 may include an input device 712 configured to allow a user to interact with any of the components of the computer system 700. The computer system 700 may also include the drive unit 716 (e.g., disk drive, optical drive). The drive unit 716 may include a computer-readable medium 722 in which one or more sets of instructions 724, e.g., software, can be embedded. Further, instructions 724 may embody one or more of the methods or logic as described. In a particular example, the instructions 724 may reside completely, or at least partially, within the memory 704 or within the processor 702 during execution by the computer system 700.

The present disclosure contemplates a computer-readable medium that includes instructions 724 or receives and executes instructions 724 responsive to a propagated signal so that a device connected to a network 726 can communicate voice, video, audio, and images or any other data over the network 726. Further, instructions 724 may be transmitted or received over the network 726 via a communication port or interface 720 or using a bus 708. The communication port or interface 720 may be a part of the processor 702 or maybe a separate component. The communication port or interface 720 may be created in software or maybe a physical connection in hardware. The communication port or interface 720 may be configured to connect with a network 726, external media, the display unit 710, or any other components in the computer system 700, or combinations thereof. The connection with the network 726 may be a physical connection, such as a wired Ethernet connection or may be established wirelessly as discussed later. Likewise, the additional connections with other components of the computer system 700 may be physical or may be established wirelessly. The network 726 may alternatively be directly connected to bus 708.

The network 726 may include wired networks, wireless networks, Ethernet AVB networks, or combinations thereof. The wireless network may be a cellular telephone network, an 802.11, 802.16, 802.20, 802.1Q or WiMax network. Further, the network 726 may be a public network, such as the Internet, a private network, such as an intranet, or combinations thereof, and may utilize a variety of networking protocols now available or later developed including, but not limited to TCP/IP based networking protocols. The system is not limited to operation with any particular standards and protocols. For example, standards for Internet and other packet-switched network transmissions (e.g., TCP/IP, UDP/IP, HTML, and HTTP) may be used.

While specific language has been used to describe the disclosure, any limitations arising on account of the same are not intended. As would be apparent to a person in the art, various working modifications may be made to the method in order to implement the inventive concept as taught herein.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein.

Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims and their equivalents.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

What is claimed is:

1. A method for mitigating physical risks associated with a user in an Internet of Things (IoT) environment, the method comprising:
monitoring multi-modal input data over a first time period, the multi-modal input data being associated with at least one multi-modal interaction of the user with at least one IoT device in the IoT environment;
identifying a change in at least one cognitive ability of the user, based on the monitored multi-modal input data;
estimating a cognitive ability index of the user, based on the identified change in the at least one cognitive ability of the user;
predicting at least one possible physical risk associated with a current user activity in the IoT environment based on a correlation of the estimated cognitive ability index with at least one cognitive health index from a plurality of cognitive health indexes;
determining at least one corrective action to avoid the predicted at least one possible physical risk associated with the current user activity; and
controlling at least one IoT device in the IoT environment to notify or perform the determined at least one corrective action such that the predicted at least one possible physical risk is mitigated.

2. The method of claim 1, further comprising:
correlating the estimated cognitive ability index with the at least one cognitive health index; and
predicting the at least one possible physical risk associated with the current user activity based on the correlation of the estimated cognitive ability index with the at least one cognitive health index.

3. The method of claim 1, wherein the at least one cognitive health index indicates at least one of a physical fitness, a mental fitness, and vulnerability of the user to perform an activity.

4. The method of claim 1, wherein the change in the at least one cognitive ability of the user corresponds to a change in cognitive health of the user to perform a task comprising at least one of a physical activity and a mental activity.

5. The method of claim 1, wherein the determined at least one corrective action corresponds to an action that, when performed by the user, results in a mitigation of the predicted at least one possible physical risk that may occur during the current user activity.

6. The method of claim 1, further comprising:
analyzing the multi-modal input data, wherein the multi-modal input data includes a plurality of parameters related to the at least one multi-modal interaction of the user;
classifying each of a corresponding parameter from the plurality of parameters related to the at least one multi-modal interaction of the user; and
generating an updated multi-modal input data based on the classification of the corresponding parameter of the plurality of parameters.

7. The method of claim 1, further comprising:
monitoring, over the first time period, at least one user activity of the user in the IoT environment; and
generating multi-modal cognitive data corresponding to the user based on the monitored multi-modal input data and the monitored at least one user activity, the multi-modal cognitive data comprising information related to the plurality of cognitive health indexes.

8. The method of claim 7, wherein the monitored at least one user activity of the user is a location-based activity corresponding to different locations within the IoT environment.

9. The method of claim 7, wherein the identifying the change in the at least one cognitive ability of the user comprises:
obtaining predefined cognitive decline criteria from a knowledge database;
converting the generated the multi-modal cognitive data into a plurality of weighted standard cognitive indexes based on the obtained predefined cognitive decline criteria;
comparing a corresponding cognitive health index of the plurality of cognitive health indexes with a corresponding weighted standard cognitive index among the plurality of weighted standard cognitive indexes; and
identifying the change in the at least one cognitive ability of the user based on a result of the comparison.

10. The method of claim 1, wherein the determining the at least one corrective action comprises:
predicting a capability of the user to handle the predicted at least one possible physical risk associated with the current user activity based on the cognitive ability index of the user;
predicting a type of the predicted at least one possible physical risk based on the predicted capability of the user to handle the predicted at least one possible physical risk; and
determining the at least one corrective action based on the predicted capability of the user to handle the predicted at least one possible physical risk and the predicted type of the at least one possible physical risk.

11. The method of claim 1, further comprising:
predicting a physical location within the IoT environment where the predicted at least one possible physical risk may occur during the current user activity based on a detection of a user's location within the IoT environment and an identification of a type of the current user activity; and
controlling at least one IoT device in the IoT environment to notify the determined at least one corrective action based on the predicted physical location to mitigate the predicted at least one possible physical risk.

12. The method of claim 1, wherein the multi-modal input data comprises information regarding cognitive health parameters associated with the user, and
wherein the cognitive health parameters correspond to at least one of Montreal Cognitive Assessment (MoCA), circadian rhythm disruption computation (CRDC), a Blood alcohol concentration (BAC) value, a percentage of water in user's body, chronic illness including arthritis, vitamin B-12 deficiency, underactive thyroid gland, and diabetic condition, a level of each of Dementia, Alzheimer's, Parkinson's, and cardiovascular diseases, and a level of blood sugar during at least one of pre-meal, post-meal, fasting.

13. A system for mitigating physical risks associated with a user in an Internet of Things (IoT) environment, the system comprising:
a memory storing instructions; and
at least one processor configured to execute the instructions to:
monitor multi-modal input data over a first time period, the multi-modal input data being associated with at least one multi-modal interaction with at least one IoT device in the IoT environment,
identify a change in at least one cognitive ability of the user, based on the monitored multi-modal input data,
estimate a cognitive ability index of the user based on the identified change in the at least one cognitive ability of the user;
predict at least one possible physical risk associated with a current user activity in the IoT environment based on a correlation of the estimated cognitive ability index with at least one cognitive health index from a plurality of cognitive health indexes,
determine at least one corrective action to avoid the predicted at least one possible physical risk associated with the current user activity, and
control at least one IoT device in the IoT environment to notify or perform the determined at least one corrective action such that the predicted at least one possible physical risk is mitigated.

14. The system of claim 13, wherein the at least one processor is further configured to execute the instructions to:
correlate the estimated cognitive ability index with the at least one cognitive health index, and
predict the at least one possible physical risk associated with the current user activity based on the correlation of the estimated cognitive ability index with the at least one cognitive health index.

15. A non-transitory computer readable medium for storing computer readable program code or instructions which are executable by a processor to perform a method for mitigating physical risks associated with a user in an Internet of Things (IoT) environment, the method comprising:
monitoring multi-modal input data over a first time period, the multi-modal input data being associated with at least one multi-modal interaction of the user with at least one IoT device in the IoT environment;
identifying a change in at least one cognitive ability of the user, based on the monitored multi-modal input data;
estimating a cognitive ability index of the user, based on the identified change in the at least one cognitive ability of the user;
predicting at least one possible physical risk associated with a current user activity in the IoT environment based on a correlation of the estimated cognitive ability index with at least one cognitive health index from a plurality of cognitive health indexes;
determining at least one corrective action to avoid the predicted at least one possible physical risk associated with the current user activity; and
controlling at least one IoT device in the environment to notify or perform the determined at least one corrective action such that the predicted at least one possible physical risk is mitigated.
